(19) United States
(12) Patent Application Publication
(10) Pub. No.: US 2026/0198823 A1
(43) Pub. Date: Jul. 16, 2026

(54) PSYCHOLOGY DETERMINATION DEVICE AND VEHICLE

(71) Applicants: SUBARU CORPORATION, Tokyo (JP); NATIONAL UNIVERSITY CORPORATION GUNMA UNIVERSITY, Gunma (JP)

(72) Inventors: Shinichi MIYAMOTO, Tokyo (JP); Sota IGUCHI, Gunma (JP); Yasushi YUMINAKA, Gunma (JP)

(21) Appl. No.: 19/445,073

(22) Filed: Jan. 9, 2026

(30) Foreign Application Priority Data
Jan. 15, 2025 (JP) 2025-005525

(51) Int. Cl. A61B 5/16; A61B 5/00; A61B 5/024; A61B 5/18

(52) U.S. Cl. CPC A61B 5/165; A61B 5/024; A61B 5/18; A61B 5/742

(57) ABSTRACT

A psychology determination device includes an acquisition unit and a psychology determination unit. The psychology determination unit is configured to sequentially generate pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data, and determine a psychological state of a user based on a Mahalanobis distance between one or more the coordinate points among the coordinate points in a feature plot and one or more coordinate points in a reference feature plot which is the feature plot serving as a reference.

### CROSS-REFERENCE TO RELATED APPLICATIONS

This application is based on and claims priority under 35 USC 119 from Japanese Patent Application No. 2025-005525 filed on January 15, 2025, the entire content of which is incorporated herein by reference.

The present disclosure relates to a psychology determination device that determines a psychological state of a user, and a vehicle including such a psychology determination device.

In vehicles, various techniques for preventing accidents have been developed. For example, Japanese Unexamined Patent Application Publication JP-A 2007-006970 discloses a technique for detecting a change in a driving state by detecting a change in fluctuation of a pulse of a driver.

An aspect of the present disclosure provides a psychology determination device. The psychology determination device includes an acquisition unit and a psychology determination unit. The acquisition unit is configured to acquire a sequence of pulse interval data indicating a pulse interval of a user. The psychology determination unit is configured to determine a psychological state of the user based on the sequence of the pulse interval data. The psychology determination unit is configured to: sequentially generate pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data; calculate, based on each of the pulse interval plots, two features among a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having the two features as a first coordinate value and a second coordinate value; and determine the psychological state of the user based on a Mahalanobis distance between one or more the coordinate points among the coordinate points in the feature plot and one or more coordinate points in a reference feature plot which is the feature plot serving as a reference.

An aspect of the present disclosure provides a vehicle. The vehicle includes a psychology determination device, and a user interface. The psychology determination device is configured to determine a psychological state of a user. The user interface is configured to notify the user of a determination result of the psychology determination device. The psychology determination device includes an acquisition unit configured to acquire a sequence of pulse interval data indicating a pulse interval of the user, and a psychology determination unit configured to determine the psychological state of the user based on the sequence of the pulse interval data. The psychology determination unit is configured to: sequentially generate pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data; calculate, based on each of the pulse interval plots, a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having predetermined two features among the first feature, the second feature, and the third feature as a first coordinate value and a second coordinate value; and determine the psychological state of the user based on a Mahalanobis distance between one or more the coordinate points among the coordinate points in the feature plot and one or more coordinate points in a reference feature plot which is the feature plot serving as a reference.

An aspect of the present disclosure provides a psychology determination device. The psychology determination device includes a memory in which a program is stored, and a processor coupled to the memory and configured to perform processing by executing the program. The processing includes: acquiring a sequence of pulse interval data indicating a pulse interval of a user; determining a psychological state of the user based on the sequence of the pulse interval data; sequentially generating pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data; calculating, based on each of the pulse interval plots, two features among a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having the two features as a first coordinate value and a second coordinate value; and determining the psychological state of the user based on a Mahalanobis distance between one or more the coordinate points among the coordinate points in the feature plot and one or more coordinate points in a reference feature plot which is the feature plot serving as a reference.

### BRIEF DESCRIPTION OF DRAWINGS

The accompanying drawings are included to provide a further understanding of the disclosure and are incorporated in and constitute a part of this specification. The drawings illustrate an embodiment and, together with the specification, serve to describe the principles of the disclosure.

FIG. 1 is a block diagram illustrating a configuration example of a vehicle according to an embodiment of the present disclosure;

FIG. 2 is a diagram illustrating an example of a Poincaré plot generated by a Poincaré plot generation unit illustrated in FIG. 1;

FIG. 3 is a diagram illustrating three features obtained from the Poincaré plot illustrated in FIG. 2;

FIG. 4 is a diagram illustrating an example of a feature plot generated by a feature plot generation unit illustrated in FIG. 1;

FIG. 5 is a diagram illustrating another example of the feature plot generated by the feature plot generation unit illustrated in FIG. 1;

FIG. 6A is a flowchart illustrating an operation example of a psychology determination device illustrated in FIG. 1;

FIG. 6B is another flowchart illustrating the operation example of the psychology determination device illustrated in FIG. 1;

FIG. 7 is a diagram illustrating an example of a Mahalanobis distance; and

FIG. 8 is a diagram illustrating another example of the Mahalanobis distance.

### DESCRIPTION OF EMBODIMENTS

It is desired that the psychological state of the user can be determined, and it is expected that the psychological state of the user can be effectively determined.

The present disclosure provides a psychology determination device that determines a psychological state of a user, and a vehicle.

Hereinafter, embodiments of the present disclosure will be described in detail with reference to the drawings. Note that the following description is directed to illustrative examples of the disclosure and not to be construed as limiting to the disclosure. Factors including, without limitation, numerical values, shapes, materials, components, positions of the components, and how the components are coupled to each other are illustrative only and not to be construed as limiting to the disclosure. Further, elements in the following example embodiments which are not recited in a most-generic independent claim of the disclosure are optional and may be provided on an as-needed basis. The drawings are schematic and are not intended to be drawn to scale. Throughout the present specification and the drawings, elements having substantially the same function and configuration are denoted with the same numerals to avoid any redundant description.

### Embodiment

### Configuration Example

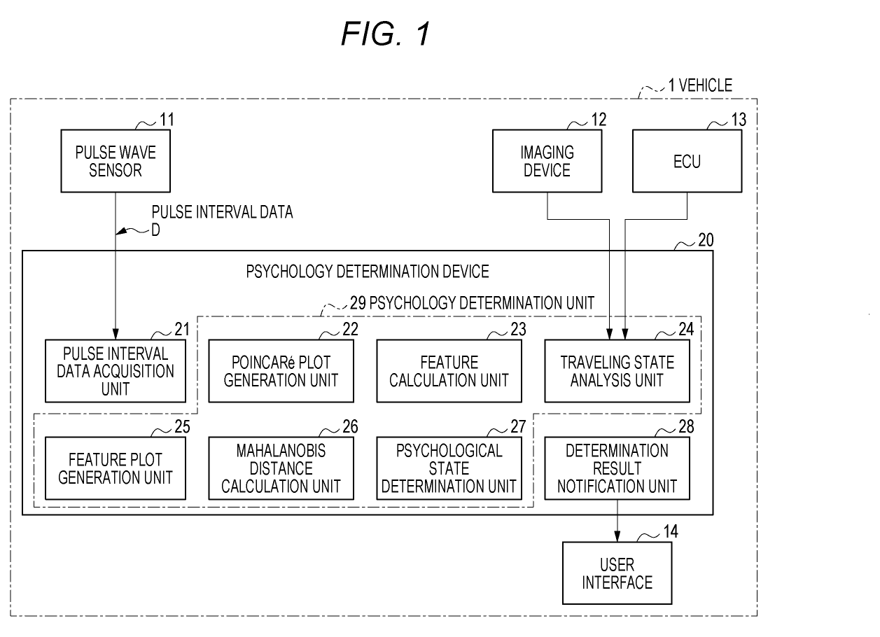

FIG. 1 illustrates a configuration example of a vehicle 1 equipped with a psychology determination device according to an embodiment. The vehicle 1 is a vehicle such as an automobile. The vehicle 1 includes a pulse wave sensor 11, an imaging device 12, an electronic control unit (ECU) 13, a psychology determination device 20, and a user interface 14.

The pulse wave sensor 11 detects a pulse wave of the driver. The pulse wave sensor 11 is attached to a portion such as an arm of the driver where the pulse wave is easily detected. The pulse wave sensor 11 measures a pulse interval called R-R interval (RRI) based on the pulse wave of the driver, and sequentially supplies data indicating the pulse interval (pulse interval data D) to the psychology determination device 20. The pulse includes a heartbeat.

The imaging device 12 generates a captured image by imaging the front of the vehicle 1. The imaging device 12 may be a monocular camera or a stereo camera. The imaging device 12 includes a lens and an image sensor. In this example, the imaging device 12 is disposed in the vicinity of an upper portion of a windshield of the vehicle 1 inside the vehicle 1. The imaging device 12 generates a series of captured images by performing an imaging operation at a predetermined frame rate (for example, 60 [fps]). The imaging device 12 supplies image data of the generated captured image to the psychology determination device 20.

The ECU 13 controls the vehicle 1, and includes, for example, a plurality of processors and memories. The ECU 13 supplies data on a traveling speed of the vehicle 1 to the psychology determination device 20.

The psychology determination device 20 determines a psychological state of the driver based on a sequence of the pulse interval data D supplied from the pulse wave sensor 11. The psychology determination device 20 includes, for example, a memory in which a program is stored and a processor coupled to the memory and configured to perform processing by executing the program. The psychology determination device 20 includes a pulse interval data acquisition unit 21, a Poincaré plot generation unit 22, a feature calculation unit 23, a traveling state analysis unit 24, a feature plot generation unit 25, a Mahalanobis distance calculation unit 26, a psychological state determination unit 27, and a determination result notification unit 28. The Poincaré plot generation unit 22, the feature calculation unit 23, the traveling state analysis unit 24, the feature plot generation unit 25, the Mahalanobis distance calculation unit 26, and the psychological state determination unit 27 constitute a psychology determination unit 29.

The pulse interval data acquisition unit 21 sequentially acquires the pulse interval data D supplied from the pulse wave sensor 11.

The Poincaré plot generation unit 22 generates a Poincaré plot P1 based on the sequence of the pulse interval data D.

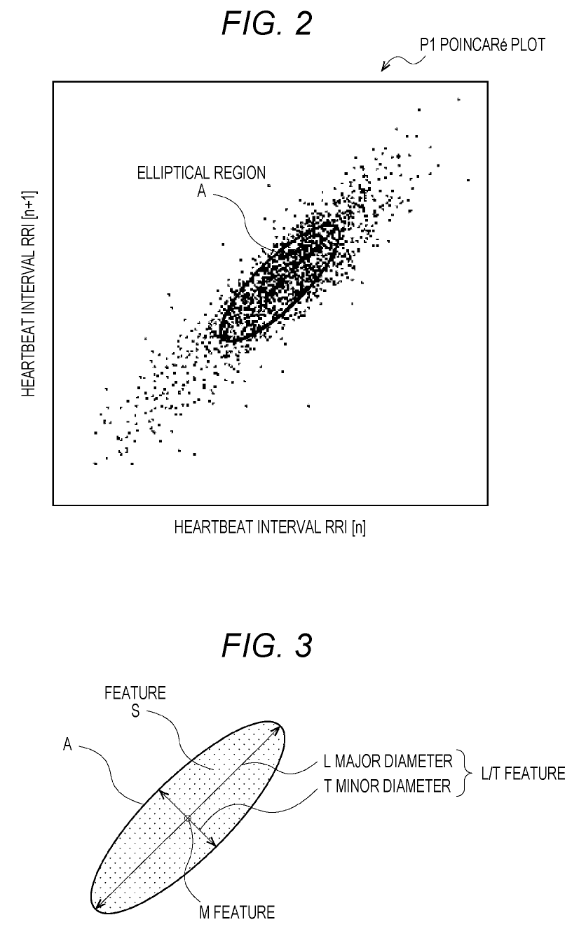

FIG. 2 illustrates an example of the Poincaré plot P1. The horizontal axis represents the n-th pulse interval, and the vertical axis represents the (n+1)-th pulse interval. Each of a plurality of points in the Poincaré plot P1 is plotted based on two adjacent pulse intervals. That is, the Poincaré plot P1 indicates a distribution of the plurality of points indicating a relationship between two adjacent pulse intervals. The Poincaré plot is also referred to as a Lorentz plot. In this example, the plurality of points are distributed to extend in a direction connecting the upper right and the lower left. That is, in this example, the Poincaré plot P1 illustrates a tendency that the (n+1)-th pulse interval becomes longer as the n-th pulse interval is long, and the (n+1)-th pulse interval becomes shorter as the n-th pulse interval is short.

The Poincaré plot generation unit 22 sequentially generates such a Poincaré plot P1 based on a latest predetermined number N2 of pieces of the pulse interval data D including the acquired pulse interval data D every time the pulse interval data acquisition unit 21 acquires a predetermined number N1 (one in this example) of pieces of the pulse interval data D. Here, the predetermined number N2 is larger than the predetermined number N1, and is, for example, about several hundreds.

The feature calculation unit 23 calculates three features (features S, M, and L/T) based on the Poincaré plot P1 every time the Poincaré plot generation unit 22 generates the Poincaré plot P1.

First, as illustrated in FIG. 2, the feature calculation unit 23 sets, based on the Poincaré plot P1, an elliptical region A by analyzing the distribution of the plurality of points. A size of the elliptical region A corresponds to a standard deviation of the distribution of the plurality of points. In the example of FIG. 2, since the plurality of points are distributed to extend in the direction connecting the upper right and the lower left, a major axis of the elliptical region A faces the direction connecting the upper right and the lower left. The feature calculation unit 23 calculates the features S, M, and L/T based on the elliptical region A.

FIG. 3 illustrates the features S, M, and L/T.

The feature S is an area value of the elliptical region A. For example, in the Poincaré plot P1, when the plurality of points are widely distributed, the standard deviation increases, and thus the feature S is a large value.

The feature M is a coordinate value of a center point of the elliptical region A in the Poincaré plot P1. Since an abscissa coordinate value and an ordinate coordinate value of the center point of the elliptical region A are equal to each other, the feature M is one of these coordinate values. For example, when the pulse interval is wide, the feature M is a large value.

The feature L/T is a value obtained by dividing a diameter of the major axis (major diameter L) of the elliptical region A by a diameter of a minor axis (minor diameter T) of the elliptical region A. For example, when a shape of the elliptical region A is close to a circle, the feature L/T is a value close to 1, and when the shape of the elliptical region A is elongated, the feature L/T is a large value.

As described above, the feature calculation unit 23 sets the elliptical region A based on the Poincaré plot P1, and calculates the three features S, M, and L/T based on the set elliptical region A. Since the Poincaré plots P1 are sequentially generated over time, the feature calculation unit 23 sequentially calculates the three features S, M, and L/T over time based on a plurality of the Poincaré plots P1. Accordingly, the feature calculation unit 23 generates a sequence of the three features S, M, and L/T.

The traveling state analysis unit 24 analyzes the image data of the captured image generated by the imaging device 12 and the data on the traveling speed of the vehicle 1 supplied from the ECU 13. Specifically, the traveling state analysis unit 24 analyzes whether a preceding vehicle is traveling in front of the vehicle 1 based on the image data of the captured image. The traveling state analysis unit 24 determines whether the traveling speed of the vehicle 1 is equal to or lower than a speed limit of a traveling road on which the vehicle 1 is traveling. For example, the traveling state analysis unit 24 may acquire information on the speed limit based on high-precision map information in a navigation system, or may acquire information on the speed limit by analyzing an image of a sign for the speed limit included in the captured image.

The feature plot generation unit 25 generates a feature plot P2 based on the sequence of the three features S, M, and L/T. The feature plot generation unit 25 generates the feature plot P2 using two of the sequences of the three features S, M, and L/T.

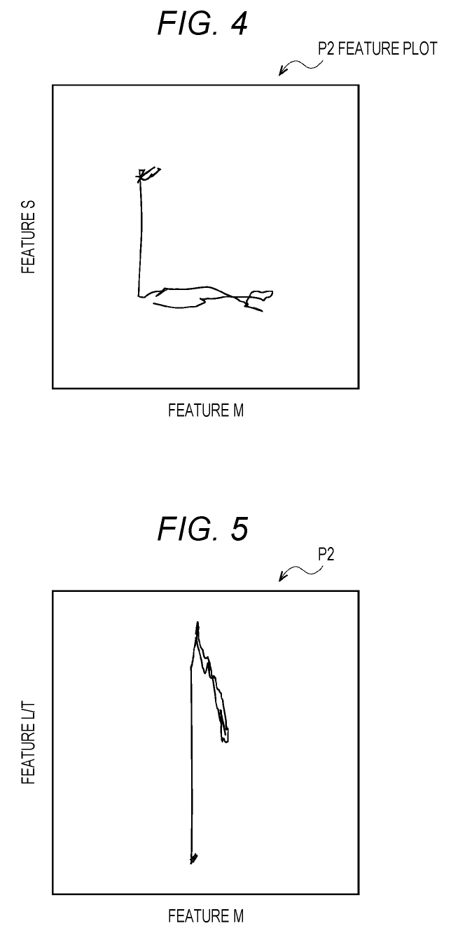

FIG. 4 illustrates an example of the feature plot P2. In this example, the feature plot generation unit 25 generates the feature plot P2 using a sequence of the features S and M among the sequences of the three features S, M, and L/T. The horizontal axis represents the feature M, and the vertical axis represents the feature S. The feature plot P2 indicates trajectories of points indicated by the features M and S. That is, the feature plot P2 indicates how the points indicated by the features M and S obtained from one Poincaré plot P1 move over time. The feature plot generation unit 25 generates such a feature plot P2 based on a predetermined number N3 of features S and the predetermined number N3 of features M. Here, the predetermined number N3 is, for example, about several hundreds.

FIG. 5 illustrates another example of the feature plot P2. In this example, the feature plot generation unit 25 generates the feature plot P2 using a sequence of the features L/T and M among the sequences of the three features S, M, and L/T. The horizontal axis represents the feature M, and the vertical axis represents the feature L/T. The feature plot P2 indicates trajectories of points indicated by the features L/T and M. The feature plot generation unit 25 generates such a feature plot P2 based on the predetermined number N3 of features L/T and the predetermined number N3 of features M.

The Mahalanobis distance calculation unit 26 calculates a Mahalanobis distance between a plurality of points in the feature plot P2 and a plurality of points in the feature plot P2 serving as a reference (reference feature plot P2R). The reference feature plot P2R is obtained, for example, when a preceding vehicle is not traveling in front of the vehicle 1 and the traveling speed of the vehicle 1 is equal to or lower than the speed limit. In other words, the reference feature plot P2R is obtained when the psychological state of the driver is, for example, a normal psychological state, not an anxious psychological state or an excited psychological state.

The psychological state determination unit 27 determines the psychological state of the driver based on the Mahalanobis distance calculated by the Mahalanobis distance calculation unit 26.

The determination result notification unit 28 performs processing of notifying the driver of information based on a determination result determined by the psychological state determination unit 27.

The user interface 14 (FIG. 1) includes a display such as a liquid crystal display device, a speaker, and the like, and can present the information to the driver. The user interface 14 presents the information to the driver based on an instruction from the psychology determination device 20.

Here, the pulse interval data acquisition unit 21 corresponds to a specific example of an "acquisition unit" in the embodiment of the present disclosure. The pulse interval data D corresponds to a specific example of "pulse interval data" in the embodiment of the present disclosure. The psychology determination unit 29 corresponds to a specific example of a "psychology determination unit" in the embodiment of the present disclosure. The predetermined number N1 corresponds to a specific example of a "first predetermined number" in the embodiment of the present disclosure. The predetermined number N2 corresponds to a specific example of a "second predetermined number" in the embodiment of the present disclosure. The Poincaré plot P1 corresponds to a specific example of a "pulse interval plot" in the embodiment of the present disclosure. The feature plot P2 corresponds to a specific example of a "feature plot" in the embodiment of the present disclosure. The reference feature plot P2R corresponds to a specific example of a "reference feature plot" in the embodiment of the present disclosure. The feature S corresponds to a specific example of a "first feature" in the embodiment of the present disclosure. The feature M corresponds to a specific example of a "second feature" in the embodiment of the present disclosure. The feature L/T corresponds to a specific example of a "third feature" in the embodiment of the present disclosure. The psychology determination device 20 corresponds to a specific example of a "psychology determination device" in the embodiment of the present disclosure. The user interface 14 corresponds to a specific example of a "user interface" in the embodiment of the present disclosure.

### Operation and Function

Next, the operation and effect of the psychology determination device 20 according to the present embodiment will be described.

### Outline of Overall Operation

First, the outline of the overall operation of the psychology determination device 20 will be described with reference to FIG. 1. The pulse interval data acquisition unit 21 sequentially acquires the pulse interval data D supplied from the pulse wave sensor 11. The Poincaré plot generation unit 22 sequentially generates the Poincaré plot P1 based on the latest predetermined number N2 of pieces of pulse interval data D including the acquired pulse interval data D every time the pulse interval data acquisition unit 21 acquires the predetermined number N1 (one in this example) of pieces of pulse interval data D. The feature calculation unit 23 calculates the three features S, M, and L/T based on the Poincaré plot P1 every time the Poincaré plot generation unit generates the Poincaré plot P1. The traveling state analysis unit 24 analyzes the image data of the captured image generated by the imaging device 12 and the data on the traveling speed of the vehicle 1 supplied from the ECU 13. The feature plot generation unit 25 generates the feature plot P2 based on the sequence of the three features S, M, and L/T. The Mahalanobis distance calculation unit 26 calculates the Mahalanobis distance between the plurality of points in the feature plot P2 and the plurality of points in the feature plot P2 serving as a reference (reference feature plot P2R). The psychological state determination unit 27 determines the psychological state of the driver based on the Mahalanobis distance calculated by the Mahalanobis distance calculation unit 26. The determination result notification unit 28 performs processing of notifying the driver of information based on the determination result determined by the psychological state determination unit 27.

### Detailed Operation

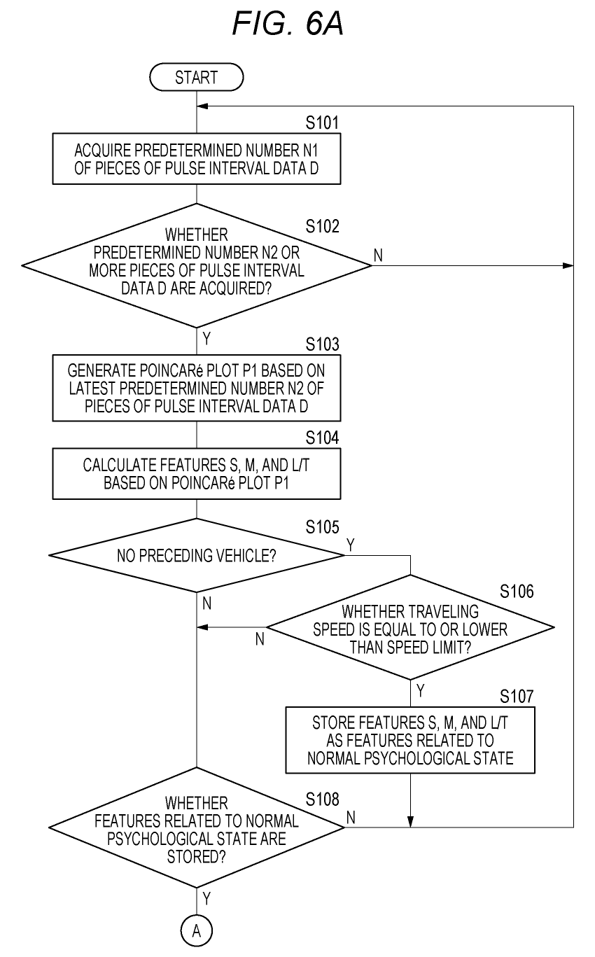

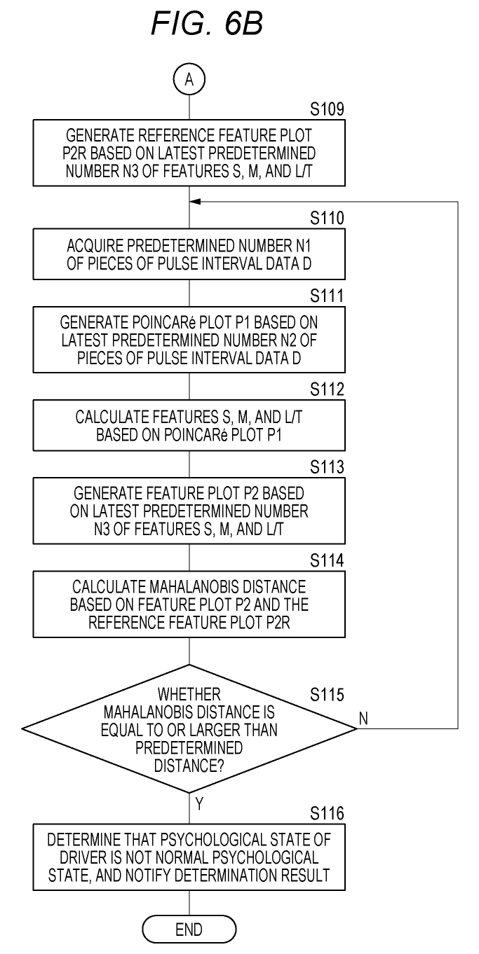

FIGS. 6A and 6B show an operation example of the psychology determination device 20. When the driver starts driving the vehicle 1, the psychology determination device 20 starts the following processing.

First, the pulse interval data acquisition unit 21 acquires the predetermined number N1 (one in this example) of pieces of pulse interval data D supplied from the pulse wave sensor 11 (step S101).

Next, the Poincaré plot generation unit 22 checks whether the predetermined number N2 or more pieces of pulse interval data D are acquired (step S102). Here, the predetermined number N2 is the number of pieces of data necessary for generating the Poincaré plot P1, and is, for example, about several hundreds. When the predetermined number N2 or more pieces of pulse interval data D are not acquired ("N" in step S102), the processing returns to step S101, and the psychology determination device 20 repeats the processing of steps S101 and S102 until the predetermined number N2 or more pieces of pulse interval data D are acquired.

When the predetermined number N2 or more pieces of pulse interval data D are acquired ("Y" in step S102), the Poincaré plot generation unit 22 generates the Poincaré plot P1 as illustrated in FIG. 2 based on the latest predetermined number N2 of pieces of pulse interval data D (step S103).

Next, the feature calculation unit 23 calculates the three features S, M, and L/T based on the Poincaré plot P1 generated in step S103 (step S104).

Next, the traveling state analysis unit 24 checks whether the preceding vehicle is traveling in front of the vehicle 1 based on the image data of the captured image generated by the imaging device 12 (step S105). When the preceding vehicle is traveling ("N" in step S105), the processing proceeds to step S108.

When the preceding vehicle is not traveling in step S105 ("Y" in step S105), the traveling state analysis unit 24 checks whether the traveling speed of the vehicle 1 is equal to or lower than the speed limit of the traveling road on which the vehicle 1 is traveling (step S106). When the traveling speed of the vehicle 1 is not equal to or lower than the speed limit ("N" in step S106), the processing proceeds to step S108.

When the traveling speed of the vehicle 1 is equal to or less than the speed limit in step S106 ("Y" in step S106), the feature calculation unit 23 stores the three features S, M, and L/T calculated in step S104 as features related to the normal psychological state (step S107). That is, since the preceding vehicle is not traveling ("Y" in step S105) and the traveling speed of the vehicle 1 is equal to or lower than the speed limit ("Y" in step S106), it is estimated that the driver is traveling at his/her pace without being affected by the surrounding vehicles. In this case, the psychological state of the driver is estimated to be, for example, a normal psychological state, not an anxious psychological state or an excited psychological state. Therefore, the feature calculation unit 23 stores the three features S, M, and L/T as features related to the normal psychological state. Then, the processing returns to step S101.

When the preceding vehicle is traveling ("N" in step S105) or when the traveling speed of the vehicle 1 is not within the speed limit ("N" in step S106), the feature calculation unit 23 checks whether the features related to the normal psychological state are stored (step S108). When the features related to the normal psychological state are not stored ("N" in step S108), the processing returns to step S101.

When the features related to the normal psychological state are stored in step S108 ("Y" in step S108), the feature plot generation unit 25 generates the feature plot P2 serving as a reference (reference feature plot P2R) based on the latest predetermined number N3 of features S, M, and L/T among the stored features related to the normal psychological state (step S109). That is, the psychology determination device 20 continuously acquires data of a plurality of pulse intervals after the driver starts driving the vehicle 1, and continuously calculates the plurality of features S, M, and L/T based on the data of the pulse intervals. Therefore, the psychology determination device 20 can store the plurality of features S, M, and L/T obtained over a long time as features related to the normal psychological state. The features S, M, and L/T can change with time even when the normal psychological state is maintained. Specifically, for example, the features S, M, and L/T obtained in the morning may be different from the features S, M, and L/T obtained in the daytime. Therefore, the feature plot generation unit 25 generates the reference feature plot P2R based on the latest predetermined number N3 of features S, M, and L/T among the stored features related to the normal psychological state. The psychology determination device 20 can determine the psychological state of the driver with higher accuracy by using the reference feature plot P2R as a comparison target.

In step S109, the feature plot generation unit 25 may generate the feature plot P2 using the sequence of the features S and M as illustrated in FIG. 4, or may generate the feature plot P2 using the sequence of the features L/T and M as illustrated in FIG. 5. Here, the predetermined number N3 is the number of pieces of data necessary for generating the feature plot P2, and is, for example, about several hundreds.

Next, similarly to step S101, the pulse interval data acquisition unit 21 acquires the predetermined number N1 (one in this example) of pieces of pulse interval data D supplied from the pulse wave sensor 11 (step S110).

Next, similarly to step S103, the Poincaré plot generation unit 22 generates the Poincaré plot P1 based on the latest predetermined number N2 of pieces of pulse interval data D (step S111).

Next, similarly to step S104, the feature calculation unit 23 calculates the three features S, M, and L/T based on the Poincaré plot P1 generated in step S111 (step S112).

The feature plot generation unit 25 generates the feature plot P2 based on the latest predetermined number N3 of features S, M, and L/T among the stored features related to the normal psychological state (step S113). When the reference feature plot P2R is generated using the sequence of the features S and M in step S109, the feature plot generation unit 25 also generates the feature plot P2 using the sequence of the features S and M in step S113. When the reference feature plot P2R is generated using the sequence of the features L/T and M in step S109, the feature plot generation unit 25 also generates the feature plot P2 using the sequence of the features L/T and M in step S113.

Next, the Mahalanobis distance calculation unit 26 calculates a Mahalanobis distance based on the feature plot P2 and the reference feature plot P2R (step S114). Specifically, the Mahalanobis distance calculation unit 26 calculates the Mahalanobis distance between the plurality of points in the feature plot P2 and the plurality of points in the reference feature plot P2R.

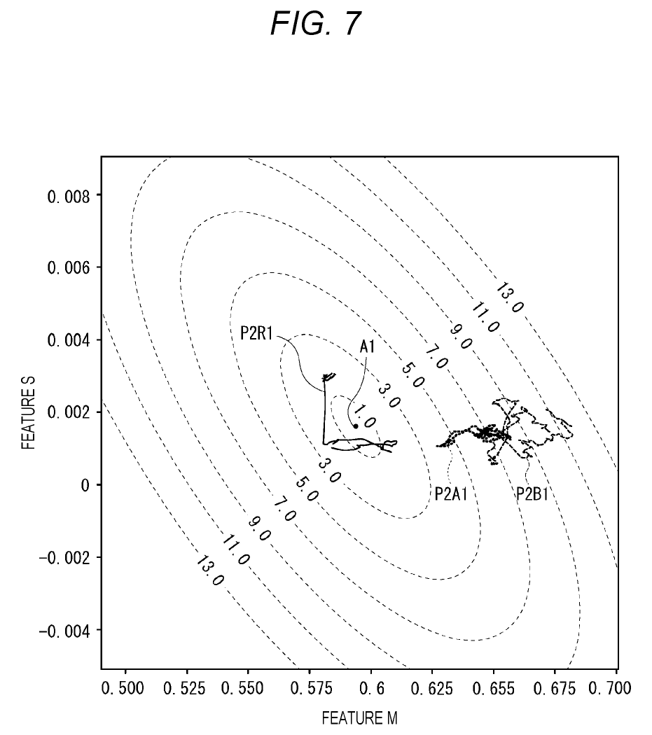

FIG. 7 illustrates an example of the Mahalanobis distance in the feature plot P2 for the features S and M. FIG. 7 illustrates the reference feature plot P2R (reference feature plot P2R1) and two feature plots P2 (feature plots P2A1 and P2B1). The feature plot P2A1 shows an example of a plot when the psychological state of the driver is a negative psychological state, and the feature plot P2B1 shows an example of a plot when the psychological state of the driver is a positive psychological state. FIG. 7 also illustrates a contour line of the Mahalanobis distance from a center point A1 of the reference feature plot P2R1, which is calculated based on a plurality of points included in the reference feature plot P2R1. As illustrated in FIG. 7, the feature plots P2A1 and P2B1 are separated from the reference feature plot P2R1. Therefore, in this example, the psychology determination device 20 can determine whether the psychological state of the driver is a normal psychological state by checking, for example, whether the Mahalanobis distance is 3 or more.

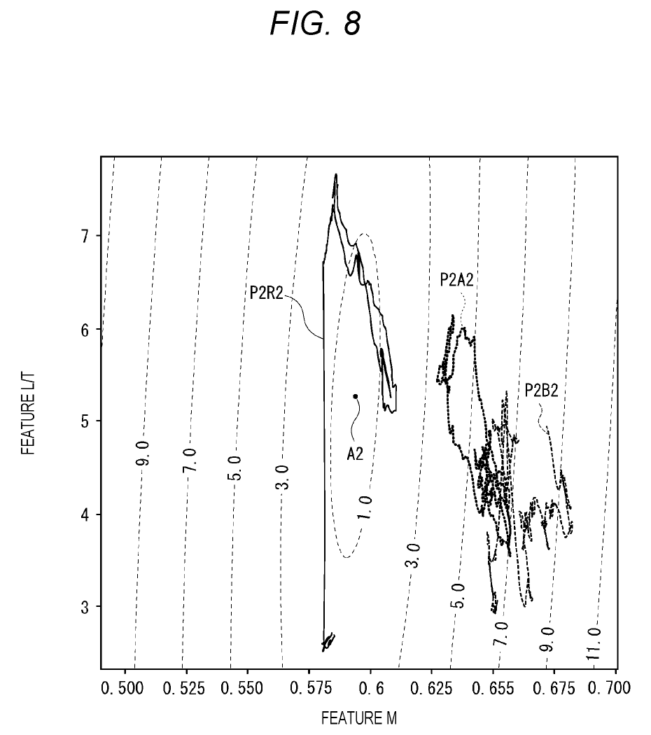

FIG. 8 illustrates an example of the Mahalanobis distance in the feature plot P2 for the features L/T and M. FIG. 8 illustrates the reference feature plot P2R (reference feature plot P2R2) and two feature plots P2 (feature plots P2A2 and P2B2). The feature plot P2A2 shows an example of a plot when the psychological state of the driver is a negative psychological state, and the feature plot P2B2 shows an example of a plot when the psychological state of the driver is a positive psychological state. FIG. 8 also illustrates a contour line of the Mahalanobis distance from a center point A2 of the reference feature plot P2R2,which is calculated based on a plurality of points included in the reference feature plot P2R2. As illustrated in FIG. 8, the feature plots P2A2 and P2B2 are separated from the reference feature plot P2R2. Therefore, in this example, the psychology determination device 20 can determine whether the psychological state of the driver is a normal psychological state by checking, for example, whether the Mahalanobis distance is 3 or more.

Next, the psychological state determination unit 27 checks whether the Mahalanobis distance calculated by the Mahalanobis distance calculation unit 26 is equal to or larger than a predetermined distance (step S115). For example, in the example of FIGS. 7 and 8, the psychological state determination unit 27 checks whether the Mahalanobis distance is 3 or more. When the Mahalanobis distance is less than the predetermined distance ("N" in step S115), the processing returns to step S110.

When the Mahalanobis distance is equal to or larger than the predetermined distance in step S115 ("Y" in step S115), the determination result notification unit 28 determines that the psychological state of the driver is not the normal psychological state, and notifies the determination result (step S116). The user interface 14 displays a message corresponding to the determination result on a screen or outputs the message by voice based on an instruction from the determination result notification unit 28. Specifically, for example, the user interface 14 notifies that the psychological state of the driver is not the normal psychological state. The user interface 14 may notify, for example, a message "please calm down". Accordingly, the driver can perform a driving operation with reference to a timely supplied message.

### This is the end of the processing.

As described above, the psychology determination device 20 includes the acquisition unit (pulse interval data acquisition unit 21) capable of acquiring the pulse interval data D including the sequence of the pulse interval of the user and the psychology determination unit 29 capable of determining the psychological state of the user based on the pulse interval data D. The psychology determination unit 29 can sequentially generate a plurality of pulse interval plots (Poincaré plots P1) by sequentially plotting, in a two-dimensional plane, a coordinate point having a first pulse interval and a second pulse interval subsequent to the first pulse interval as a first coordinate value and a second coordinate value, respectively, based on a latest second predetermined number (predetermined number N2) of pieces of pulse interval data D every time the acquisition unit (pulse interval data acquisition unit 21) acquires a first predetermined number (predetermined number N1) of pieces of pulse interval data D, calculate, based on each of the plurality of pulse interval plots (Poincaré plots P1), two features among a first feature (feature S) indicating an area of the elliptical region A defined by a plurality of coordinate points, a second feature (feature M) indicating a center position of the elliptical region A, and a third feature (L/T) indicating a ratio of the major diameter L and the minor diameter T of the elliptical region A and generate the feature plot P2 by plotting, in a two-dimensional plane, a coordinate point having the two features as a first coordinate value and a second coordinate value, and determine the psychological state of the user based on a Mahalanobis distance between one or more coordinate points among the plurality of coordinate points in the feature plot P2 and one or more coordinate points in the reference feature plot P2R which is the feature plot serving as a reference. In the psychology determination device 20, as illustrated in FIGS. 7 and 8, the feature plot P2 varies depending on the psychological state of the user. Therefore, the psychology determination device 20 can determine the psychological state of the user based on the feature plot P2.

In the psychology determination device 20, the reference feature plot P2R is generated based on the pulse interval data D when the psychological state of the user is the normal psychological state. The psychology determination unit 29 can determine whether the psychological state of the user is the normal psychological state based on the Mahalanobis distance. As described above, in the psychology determination device 20, based on the feature plot P2 of the user and the reference feature plot P2R of the user, it is determined whether the psychological state of the user is the normal psychological state similar to that when the reference feature plot P2R is obtained, and thus it is possible to determine whether the psychological state of the user is the normal psychological state by a simple method.

### Effect

As described above, in the present embodiment, the acquisition unit capable of acquiring the pulse interval data D including the sequence of the pulse interval of the user and the psychology determination unit capable of determining the psychological state of the user based on the pulse interval data are provided. The psychology determination unit can sequentially generate a plurality of pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data, calculate, based on each of the plurality of pulse interval plots, two features among a first feature indicating an area of an elliptical region defined by a plurality of the coordinate points, a second feature indicating a center position of the elliptical region, and a third feature indicating a ratio of a major diameter and a minor diameter of the elliptical region and generate a feature plot by plotting, in a two-dimensional plane, a coordinate point having the two features as a first coordinate value and a second coordinate value, and determine the psychological state of the user based on a Mahalanobis distance between one or more the coordinate points among the plurality of coordinate points in the feature plot and one or more coordinate points in a reference feature plot which is the feature plot serving as a reference. Accordingly, the psychological state of the user can be determined.

In the present embodiment, the reference feature plot is generated based on the pulse interval data when the psychological state of the user is a normal psychological state. The psychology determination unit can determine whether the psychological state of the user is the normal psychological state based on the Mahalanobis distance. Accordingly, it is possible to determine whether the psychological state of the user is the normal psychological state by a simple method.

Although the present technology has been described above with reference to the embodiments, the present technology is not limited to these embodiments and the like, and various modifications can be made.

For example, in the above-described embodiment, the predetermined number N1 is set to "1", and the Poincaré plot generation unit 22 sequentially generates the Poincaré plot P1 based on the latest predetermined number N2 of pieces of pulse interval data D every time the pulse interval data acquisition unit 21 acquires one piece of pulse interval data D. However, the present disclosure is not limited thereto. The predetermined number N1 may be a number other than 1. For example, every time the pulse interval data acquisition unit 21 acquires two pieces of pulse interval data D, the Poincaré plot generation unit 22 may sequentially generate the Poincaré plot P1 based on the latest predetermined number N2 of pieces of pulse interval data D.

For example, in the above-described embodiment, the present technology is applied to a vehicle, but the present technology is not limited thereto, and may be applied to various moving objects instead.

The effects described in the present specification are merely examples and are not limited, and there may be other effects.

Further, the present disclosure can take the following aspects.

(1) A psychology determination device including:

an acquisition unit configured to acquire a sequence of pulse interval data indicating a pulse interval of a user; and

a psychology determination unit configured to determine a psychological state of the user based on the sequence of the pulse interval data, in which

the psychology determination unit is configured to

sequentially generate pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data,

calculate, based on each of the pulse interval plots, a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having predetermined two features among the first feature, the second feature, and the third feature as a first coordinate value and a second coordinate value, and

determine the psychological state of the user based on a Mahalanobis distance between one or more the coordinate points among the coordinate points in the feature plot and one or more coordinate points in a reference feature plot which is the feature plot serving as a reference.

(2) The psychology determination device according to (1), in which

the reference feature plot is generated based on the pulse interval data when the psychological state of the user is a normal psychological state, and

the psychology determination unit is configured to determine whether the psychological state of the user is the normal psychological state based on the Mahalanobis distance.

(3) The psychology determination device according to (1) or (2), in which

the two features include the second feature.

(4) A vehicle including:

a psychology determination device configured to determine a psychological state of a user; and

a user interface configured to notify the user of a determination result of the psychology determination device, in which

the psychology determination device includes

an acquisition unit configured to acquire a sequence of pulse interval data indicating a pulse interval of the user, and

a psychology determination unit configured to determine the psychological state of the user based on the sequence of the pulse interval data, and

the psychology determination unit is configured to

sequentially generate pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data,

calculate, based on each of the pulse interval plots, a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having predetermined two features among the first feature, the second feature, and the third feature as a first coordinate value and a second coordinate value, and

determine the psychological state of the user based on a Mahalanobis distance between one or more the coordinate points among the coordinate points in the feature plot and one or more coordinate points in a reference feature plot which is the feature plot serving as a reference.

(5) The vehicle according to (4), in which

the psychology determination unit is configured to

analyze traveling-related information related to a traveling state of the vehicle,

generate the reference feature plot when the traveling-related information satisfies a predetermined condition, and

generate the feature plot when the traveling-related information does not satisfy the predetermined condition.

(6) A psychology determination device including:

a memory in which a program is stored; and

a processor coupled to the memory and configured to perform processing by executing the program, in which

the processing includes:

acquiring a sequence of pulse interval data indicating a pulse interval of a user;

determining a psychological state of the user based on the sequence of the pulse interval data,

sequentially generating pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data;

calculating, based on each of the pulse interval plots, two features among a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having the two features as a first coordinate value and a second coordinate value; and

determining the psychological state of the user based on a Mahalanobis distance between one or more the coordinate points among the coordinate points in the feature plot and one or more coordinate points in a reference feature plot which is the feature plot serving as a reference.

What is claimed is:

1. A psychology determination device comprising:
an acquisition unit configured to acquire a sequence of pulse interval data indicating a pulse interval of a user; and
a psychology determination unit configured to determine a psychological state of the user based on the sequence of the pulse interval data,
wherein the psychology determination unit is configured to
sequentially generate pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data,
calculate, based on each of the pulse interval plots, two features among a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having the two features as a first coordinate value and a second coordinate value, and
determine the psychological state of the user based on a Mahalanobis distance between one or more the coordinate points among the coordinate points in the feature plot and one or more coordinate points in a reference feature plot which is the feature plot serving as a reference.

2. The psychology determination device according to claim 1,
wherein the reference feature plot is generated based on the pulse interval data when the psychological state of the user is a normal psychological state, and
wherein the psychology determination unit is configured to determine whether the psychological state of the user is the normal psychological state based on the Mahalanobis distance.

3. The psychology determination device according to claim 1,
wherein the two features comprise the second feature.

4. A vehicle comprising:
a psychology determination device configured to determine a psychological state of a user; and
a user interface configured to notify the user of a determination result of the psychology determination device,
wherein the psychology determination device comprises
an acquisition unit configured to acquire a sequence of pulse interval data indicating a pulse interval of the user, and
a psychology determination unit configured to determine the psychological state of the user based on the sequence of the pulse interval data, and
wherein the psychology determination unit is configured to
sequentially generate pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data,
calculate, based on each of the pulse interval plots, a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having predetermined two features among the first feature, the second feature, and the third feature as a first coordinate value and a second coordinate value, and
determine the psychological state of the user based on a Mahalanobis distance between one or more the coordinate points among the coordinate points in the feature plot and one or more coordinate points in a reference feature plot which is the feature plot serving as a reference.

5. The vehicle according to claim 4,
wherein the psychology determination unit is configured to
analyze traveling-related information related to a traveling state of the vehicle,
generate the reference feature plot when the traveling-related information satisfies a predetermined condition, and
generate the feature plot when the traveling-related information does not satisfy the predetermined condition.

6. A psychology determination device comprising:
a memory in which a program is stored; and
a processor coupled to the memory and configured to perform processing by executing the program,
wherein the processing comprises:
acquiring a sequence of pulse interval data indicating a pulse interval of a user;
determining a psychological state of the user based on the sequence of the pulse interval data,
sequentially generating pulse interval plots by sequentially plotting, in a two-dimensional plane, a coordinate point having a first pulse interval and a second pulse interval subsequent to the first pulse interval as a first coordinate value and a second coordinate value, respectively, based on a latest second predetermined number of pieces of the pulse interval data every time the acquisition unit acquires a first predetermined number of pieces of the pulse interval data;
calculating, based on each of the pulse interval plots, two features among a first feature indicating an area of an elliptical region defined by the coordinate points, a second feature indicating a center position of the elliptical region, and a third feature indicating a ratio of a major diameter and a minor diameter of the elliptical region, and generate a feature plot by plotting, in a two-dimensional plane, a coordinate point having the two features as a first coordinate value and a second coordinate value; and
determining the psychological state of the user based on a Mahalanobis distance between one or more the coordinate points among the coordinate points in the feature plot and one or more coordinate points in a reference feature plot which is the feature plot serving as a reference.
